(19) United States
(12) Patent Application Publication
(10) Pub. No.: US 2013/0095536 A1
(43) Pub. Date: Apr. 18, 2013

(54) CARBON DIOXIDE CAPTURE BOX

(71) Applicant: Dinabandhu Sahoo, New Delhi (IN)

(72) Inventor: Dinabandhu Sahoo, New Delhi (IN)

(21) Appl. No.: 13/704,176

(22) PCT Filed: Jun. 10, 2011
(86) PCT No.: PCT/IN2011/000389

§ 371 Date: Dec. 13, 2012

(30) Foreign Application Priority Data
Jun. 16, 2010 (IN) 1395/DEL/2010

(51) Int. Cl. C12M 1/00

(52) U.S. Cl. CPC C12M 43/04
USPC 435/134; 435/289.1; 435/292.1; 435/163

(57) ABSTRACT

The present subject matter relates to a system comprising a first unit that generates a polluted air comprising carbon dioxide, and a second unit mounted on the first unit. The second unit comprises a solution comprising at least one alga suspended in a solvent to purify the polluted air. The solution comprising the at least one alga is agitated upon operation of the first unit. The system further comprises a channel to guide the polluted air from the first unit to the second unit.

### TECHNICAL FIELD

The present subject matter relates to a carbon dioxide capture box, and a system comprising a carbon dioxide capture box, according to the preambles of independent claims.

### BACKGROUND

“Global Warming” is a major topic of concern throughout the world. Global warming is caused due to the presence of greenhouse gases, mainly consisting of carbon dioxide (CO₂). Intensive fossil-fuel burning and deforestation over the years have increased atmospheric CO₂ by almost 40% above pre-industrial values. The increasing CO₂ level has increased the average global temperature, and hence has disturbed the climatic conditions globally. The major source of CO₂ emissions is from power plants, industries, agricultural waste and transportation. The transport sector contributes substantially to the global CO₂ level and within the transport sector, road transport automobiles, for example scooters, motorcycles, cars, jeeps, vans, buses, trucks etc., are major contributors of the emission. The biological, social and economic consequences of enhanced CO₂ in the atmosphere are well known. Unless some mitigating measures are implemented to control or reduce CO₂ levels, this will create more problems.

Various measures are known that are implemented to control CO₂ levels. One of the conventional systems for this purpose is a photo-bioreactor, which is an algae-based system that converts CO₂ into useful product(s). Working of a photo-bioreactor is commonly known. It requires a solution containing algae, which is aerated by the agitation of the solution. The algae capture CO₂ and convert it into carbohydrate in the presence of light through a process called photosynthesis. Further, conventionally, photo-bioreactors are known to grow algae in the laboratory scale and industrial scale for various purposes.

The conventional photo-bioreactors are usually big in size and volume that occupy large spaces and involve high cost for installation and operation. They require light sources (both natural and artificial) to provide light to the algae, which is essential to convert CO₂ into useful products, mainly carbohydrates. Further, in the conventional photo-bioreactors, the algae solution has to be constantly agitated for the aeration of algae. This is essential for efficient working of the photo-bioreactor. The requirement of artificial light and constant mechanical agitation of algae solution makes the photo-bioreactor assembly energy consuming, complex and costly.

As stated above, transport sector is one of the major source of CO₂ emissions. This includes not only surface (road) transport such as scooters, motorcycles, cars, buses, trucks etc., but also sea and air transport, such as ships and airplanes. All the motor driven vehicles running on fossil fuels emit CO₂ along with other pollutants. Measures are known and implemented to control (minimize) vehicular pollution. For example, the automobiles are fitted with catalytic converters to minimize the vehicular emissions. However, these catalytic converters do not reduce the CO₂ emissions substantially. The other conventional measures also do not exclusively reduce the CO₂ emitted by the motorized vehicles. Furthermore, the conventional measures to control or reduce CO₂ emissions are expensive, and most of them are not practical to be implemented in mass-scale.

### SUMMARY

The subject matter disclosed herein describes a system comprising a first unit that generates a polluted air comprising carbon dioxide, and a second unit mounted on the first unit. The second unit comprises a solution comprising at least one alga suspended in a solvent to purify the polluted air. The solution comprising the at least one alga is agitated upon operation of the first unit. The system further comprises a channel to guide the polluted air from the first unit to the second unit.

The subject matter disclosed herein further describes a carbon dioxide capture box comprising at least one purifying unit, partially filled with a solution comprising at least one alga suspended in a solvent, to purify a polluted air. The polluted air comprises carbon dioxide which is a major Green House Gas. The purifying unit is a housing that comprises a transparent surface to provide sunlight to the at least one alga. The carbon dioxide capture box is mounted on a vehicle. The solution comprising the at least one alga agitates upon movement of the vehicle.

These and other features, aspects, and advantages of the present subject matter will become better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

The novel features of the subject matter are set forth in the appended claims hereto. The subject matter itself, however, as well as a preferred mode of use, further objectives, and advantages thereof, will best be understood by reference to the following detailed description of illustrative embodiments when read in conjunction with the accompanying drawing, and wherein:

FIG. 1 illustrates a block diagram of a system including a carbon dioxide capture box, according to the present subject matter.

FIG. 2 illustrates a flow diagram to convert the algal bio-mass produced by consumption of carbon dioxide in the carbon dioxide capture box, according to the present subject matter, into bio-fuel and other products.

### DETAILED DESCRIPTION

Photo-bioreactors are systems that are known to grow algae in the presence of light. They are installed in the laboratories and algal industries to grow algae. Further, photo-bioreactors are utilized to capture carbon dioxide (CO₂) from ambience and convert the captured CO₂ to useful products. These photo-bioreactors are installed near power plants and industrial areas and they occupy large installation spaces and also incur high cost of operation. Further, the algae solution needs light input and substantial agitation for its aeration, for the conversion of CO₂ into the useful products. The conventional photo-bioreactors utilize both artificial light and natural light for the purpose, and the algae solution is agitated through mechanical means. Transport sector that includes surface, air and sea based motorized vehicles, operating on fossil fuels, are major sources of CO₂ in the atmosphere. Increasing amount of CO₂ in air has contributed to “Global Warming”. In order to address the issue of global warming, it is essential to control CO₂ emissions from the motorized vehicles operating on fossil fuels, such as petrol, gasoline, diesel, petroleum gas, compressed natural gas (CNG) etc. Conventional measures taken, including the photo-bioreactors, for suppressing pollutants and harmful gases, including CO₂ do not consider controlling the vehicular CO₂ emissions exclusively. Thus there is a need of a system or a device that can capture CO₂ emitted from the motorized vehicles and control its release in the atmosphere.

The present subject matter relates to a carbon dioxide capture box, particularly a portable carbon dioxide capture box, and a system including a carbon dioxide capture box that captures CO₂ from vehicular exhaust and/or ambient air and converts the captured CO₂ to useful products. In this way, emission of CO₂, which is a major “Green House Gas”, in atmosphere can be substantially reduced. For the purpose of simplicity, the carbon dioxide capture box may be referred as capture box hereinafter in the specification.

The carbon dioxide capture box, according to the present subject matter, is portable and can be mounted on a motorized vehicle, non-motorized vehicle or any moving object. The capture box includes at least one purifying unit. The purifying unit is a housing that is partially filled with a solution comprising at least one alga suspended in a suitable solvent. The housing of the purifying unit has at least one surface made of transparent material to allow sunlight (natural light) and/or artificial light reach the algae solution. Further, the purifying unit includes a plurality of inlets to receive CO₂ emitted from the vehicle on which the capture box is mounted and/or from ambience, and also comprises a plurality of outlets to discharge the purified air (oxygen) and/or the algae solution. In case the capture box is mounted on the motorized vehicle that emits CO₂ upon its operation, a channel is provided that links the exhaust pipe (or the discharge end) of the vehicle and the capture box. The channel is configured to capture CO₂ emitted from the exhaust pipe of the vehicle and guide the captured CO₂ to the purifying unit via one of the inlets. The channel is fitted with an exhaust collector at one of its ends near the exhaust pipe of the vehicle. The exhaust collector is configured to capture the maximum amount of pollutants, including CO₂, emitted from the vehicle.

The working of the carbon dioxide capture box mounted on a motorized vehicle, according to the present subject matter, is as follows. The motorized vehicle upon its operation emits pollutants, including CO₂ that are captured and guided into the purifying unit of the carbon dioxide capture box. The algae in the purifying unit absorb the CO₂ content. The algae also absorb the CO₂ received from the ambience via other inlet(s). Further, the algae may also absorb other polluting gases, such as oxides of sulfur and/or oxides of nitrogen. The algae solution receives sunlight (natural light) via at least one transparent surface of the purifying unit. The algae solution may also receive artificial light from the at least one transparent surface. Further, the algae solution is agitated upon movement or motion of the vehicle. The agitation causes aeration of the algae solution. The algae present in the solution, in the presence of light, convert CO₂ into purified air, i.e. oxygen, and other useful products. The algae may also convert the other polluting gases into useful products. The purified air get collected above the algae solution as the purifying unit is partially filled. This purified air can be discharged into atmosphere via one of the outlets of the purifying unit or can be supplied to the interior or passenger(s) of the vehicle. Once the algae solution is saturated (after optimum algal growth), the alga can be discharged or taken out via one of the outlets of the purifying unit, and fresh algae solution can be added to the purifying unit.

The carbon dioxide capture box, according to the present subject matter, can be made aerodynamic to reduce drag coefficient of the carbon dioxide capture box-vehicle system upon its installation.

Further, in case the portable carbon dioxide capture box, according to the present subject matter, is mounted on a non-motorized vehicle or any other moving object, the purifying unit receives/captures CO₂ and/or other polluting gases, such as oxides of sulfur and nitrogen, from ambience and converts them to purified air and other useful products. In this case, the vehicle or the moving object upon its operation or movement provides agitation to the algae solution to aerate the algae. The algae solution receives sunlight (natural light) and/or artificial light from the at least one transparent surface of the purifying unit. In the presence of light the aerated algae converts the captured CO₂ to useful products.

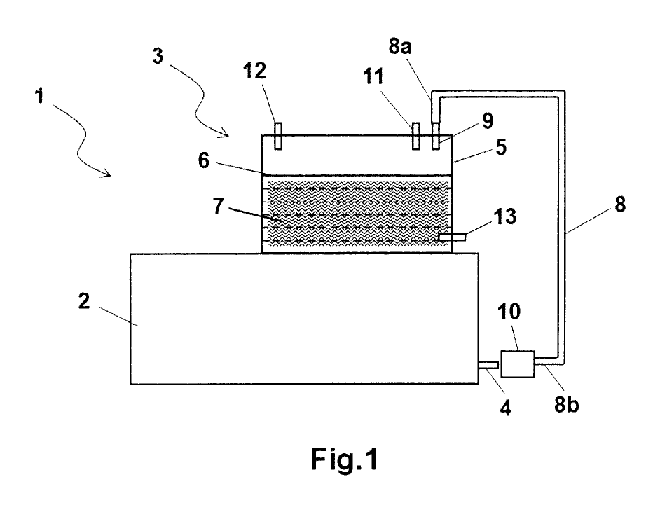

FIG. 1 illustrates a block diagram of a system 1, according to the present subject matter, including a first unit 2 that generates a polluted air including CO₂, and a second unit 3 mounted on the first unit 2. The first unit 2 includes a discharge end 4 via which pollutants, including CO₂, generated upon operation of the first unit 2, get ejected. The discharge end 4 can be termed as ‘exhaust channel’ 4 and both the terms can be used interchangeably in the specification.

The second unit 3 includes at least one purifying unit 5. The purifying unit 5 is a housing partially filled with an algae solution or suspension 6. The algae solution 6 includes at least one alga 7 suspended in a suitable solvent. The algae solution 6 is agitated upon operation of the first unit 2. Further, at least one surface (not shown) of the purifying unit 5 is made transparent to let sunlight into the purifying unit 5. The sunlight is provided to the alga 7, which is necessary for conversion of CO₂ into a useful product. In an embodiment, a portion of purifying unit 5 can be made transparent to provide sunlight to the alga 7. Further, in an embodiment, the purifying unit 5 may receive sunlight (natural light) and/or artificial light via the at least one transparent surface.

The system 1, according to the present subject matter, further includes a channel 8 to guide the pollutants emitted by the first unit 2 into the at least one purifying unit 5 of the second unit 3. The channel 8 includes at least two ends, a first end 8a and a second end 8b that link the first unit 2 with the purifying unit 5 of the second unit 3. As shown in FIG. 1, the channel 8 at the first end 8a is connected to an inlet 9 (first inlet) of the purifying unit 5. Further, the channel 8 is connected to an exhaust collector 10 at the second end 8b. The exhaust collector 10 collects the pollutants, i.e. the polluted air, preferably CO₂, emitted by the first unit 2. The exhaust collector 10 is fitted with a filtering system (not shown) to filter out suspended particulate matter (SPM), and thus substantially preventing the SPM reaching the purifying unit 5.

In an embodiment, a filtering system (not shown) is fitted in the channel 8 to filter out other particles so that CO₂ and some gases reach the purifying unit 5.

In the preferred embodiment, the exhaust collector 10 is positioned close to the discharge end 4 of the first unit 2, but not in direct contact with the discharge end 4. The exhaust collector 10 is configured in such a way that maximum amount of pollutants from the discharge end 4 enter the exhaust collector 10 and get guided to the purifying unit 5.

Further, according to the preferred embodiment, the purifying unit 5 includes another inlet 11 (second inlet) for receiving a polluted air, including CO₂, from atmosphere. Further in the preferred embodiment, the purifying unit 5 includes a first outlet 12 to discharge a purified air (oxygen) and/or vapour generated in the purifying unit 5, and a second outlet 13 to discharge the algae solution 6 from the purifying unit 5.

In an embodiment, the purified air (oxygen) discharged from the purifying unit 5 is supplied inside the first unit 2. The purified air can be supplied to a passenger cabin of the first unit 2 and/or to at least one passenger of the first unit 2.

In an embodiment, the purified air (oxygen) discharged from the purifying unit 5 is supplied in the first unit 2 through the first outlet 12.

In an embodiment, the purifying unit 5 includes another outlet (not shown) to discharge and supply the purified air (oxygen) to the passenger cabin of the first unit 2 or to at least one passenger of the first unit 2.

Further, for the second unit 3, the purifying unit 5 is made from any one from acrylic, polycarbonate, polyvinyl chloride, recycle plastic, polyethylene, nylon, fiber-glass or any other material used for the same purpose.

Further, according to the preferred embodiment, the system 1 includes at least one monitoring unit (not shown) to measure parameters such as temperature, pH and density of the algae solution 6. The at least one monitoring unit may measure light intensity in the purifying unit 5. For measuring these parameters one or more probes of the respective monitoring units can be inserted in the purifying unit 5. The insertion can be from one of the inlets and/or outlets of the purifying unit 5. In an embodiment, the purifying unit 5 may include other inlets, particularly for insertion of the probe(s). For example, a thermometer can be inserted through another inlet (third inlet, not shown) of the purifying unit 5 to measure the temperature of the algae solution 6.

Further, in an embodiment, the first unit 2 can be a motorized vehicle running on fossil fuel or CNG that emits pollutants including CO₂.

In an embodiment, the first unit 2 can be a scooter, a motorcycle, a car, a jeep, an SUV, a transport container, a bus, a truck, a ship.

Further, in an embodiment, the first unit 2 can be a non-motorized vehicle, such as cycle, rickshaw, trolley or cart. In this case, the second unit 3 may not be linked or connected to the first unit 2 via the channel 8. The second unit 3, in this case, is portable that captures CO₂ from atmosphere and convert the captured CO₂ to purified air and/or useful products.

Further, in an embodiment, the first unit 2 can be a movable object, and the second unit 3, which is portable, is mounted on the movable object that can provide, upon its movement, a substantial agitation to the algae solution 6.

In an embodiment, the alga 7 can be strains or varieties or species of any one from Botryococcus, Chlorella, Scenedesmus, Ankistrodesmus, Nostoc, Anabaena, Oscillatoria etc. The alga 7 can be any other suitable alga used for the same purpose.

Further, in the preferred embodiment, the second unit 3 purifies the polluted air including CO₂.

In an embodiment, the second unit 3 may purify an oxide of carbon and/or an oxide of nitrogen and/or an oxide of sulfur received from the first unit 2 and/or atmosphere.

In an embodiment, the second unit 3 is mounted on the first unit 2 through fasteners.

In an embodiment, the second unit 3 is aerodynamic and portable. In an embodiment, the purifying unit 5 can be of any shape that promotes the portability and aerodynamics of the second unit 3. Further in an embodiment, the purifying unit 5 may include a tank or a chamber to hold the algae solution 6. The tank or the chamber can be of any shape that promotes the portability and aerodynamics of the second unit 3.

In an embodiment, the exhaust collector 10 can be a conical shaped collector or a collector of any other shape.

According to an alternate embodiment, the exhaust collector 10 can be connected to the discharge end 4 or exhaust channel 4 of the first unit 2 to take up substantially all the pollutants ejected from the exhaust channel 4.

The second unit 3, according to the present subject matter, is a carbon dioxide capture box 3, and the first unit 2, shown in FIG. 1, is a vehicle 2. The above description of FIG. 1 in the specification holds true for the carbon dioxide capture box 3 as second unit 3 and the vehicle 2 as first unit 2.

Thus, as shown in FIG. 1, the carbon dioxide capture box 3 is mounted on the vehicle 2. The carbon dioxide capture box 3 is portable. In the preferred embodiment of the present subject matter, the vehicle 2 is a motorized vehicle running on fossil fuels or CNG that emits pollutants including CO₂ upon its operation. In an embodiment, the exhaust channel 4 is a conventional tail pipe or exhaust pipe of a motorized vehicle.

In an embodiment, the vehicle 2 can be a scooter, a motorcycle, a car, a jeep, an SUV, a transport container, a bus, a truck or a ship.

The carbon dioxide capture box 3 includes the purifying unit 5 partially filled with the algae solution 6, and includes the channel 8 to guide the polluted air emitted by the vehicle 2 into the purifying unit 5.

In an embodiment, the vehicle 2 can be a non-motorized vehicle, such as cycle, rickshaw, trolley or cart. In this case, the carbon dioxide capture box 3 is portable that captures CO₂ from ambience.

Further, in an embodiment, instead of a vehicle 2, the carbon dioxide capture box 3, which is portable, can be mounted on any movable object that can provide a substantial agitation to the algae solution 6.

Other features of the carbon dioxide capture box 3 mounted on the vehicle 2 are considered to be same as described earlier for the second unit 3 mounted on the first unit 2.

Further, in an embodiment, information of the measured parameters measured by the at least one monitoring unit is displayed. In an embodiment, said information is displayed to a passenger driving the vehicle 2.

The carbon dioxide capture box 3, according to the present subject matter, has the following advantages. The carbon dioxide capture box is portable or mobile and does not occupy a large space. It can be mounted on any vehicle or any moving object. Further, the carbon dioxide capture box 3, according to the present subject matter, utilizes natural light, i.e. sunlight, as light source for conversion of CO₂ into useful products and the algae solution 6 is naturally agitated by the motion of the vehicle or the object onto which it is mounted. Further advantage is that the carbon dioxide capture box 3, according to the present subject matter, captures CO₂ emitted from the vehicles directly and converts it into useful products and purified air. With this the emission of CO₂ (green house gas) into the atmosphere is substantially prevented. Further, the portable/mobile carbon dioxide capture box 3, according to the present subject matter, can be configured to maintain aerodynamics of carbon dioxide capture box-vehicle system. This helps in keeping the drag coefficient of the system in check.

Further, the carbon dioxide capture box 3, according to the present subject matter, is capable of receiving artificial light from external sources, such as street-lights, lights in parking places, lamps etc., in addition to sunlight, for conversion of CO₂ into useful products in night time and/or in the absence of sunlight. Thus, the capture box 3 does not need an artificial light source installed on the box 3. Further, the carbon dioxide capture box 3, according to the present subject matter, utilizes heat from the vehicular exhausts for substantially keeping the temperature of the algae solution 6 to optimal level for its conversion into useful products. With this a heating source need not be installed in the capture box 3. The above advantages result in substantial reduction of energy consumption during the operation of the carbon dioxide capture box 3.

The portable aerodynamic carbon dioxide capture box 3, according to the present subject matter, can be implemented in transport related industries, particularly for green automobiles and ships.

In an embodiment, the carbon dioxide capture box 3 is a portable photo-bioreactor.

The carbon dioxide capture box 3, according to the present subject matter, converts the captured CO₂ into purified air (oxygen) and other useful products such as bio-mass. A typical bio-mass produced is Algal that can be further processed to produce bio-fuel such as bio-diesel, bio-ethanol etc., and various other products such as fertilizers, useful chemicals etc.

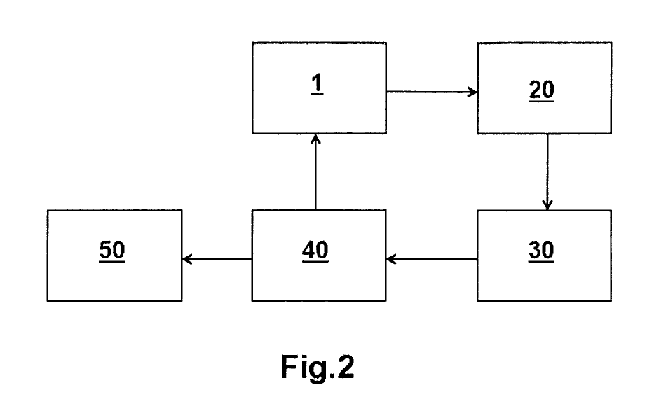

FIG. 2 illustrates a flow diagram, according to an embodiment of the present subject matter, to convert the bio-mass into bio-fuel and other products. The bio-mass produced in the carbon dioxide capture box 3 of the system 1 is taken out from the carbon dioxide capture box 3 and a fresh alga 7 is added. The bio-mass is taken to a receiving unit 20. From the receiving unit 20, the bio-mass is taken to a conversion unit 30 for conversion of the bio-mass into bio-fuel such as bio-diesel, bio-ethanol etc., and/or other products such as fertilizers, chemicals etc. The bio-fuel produced by the conversion unit 30 can be taken to a supplying unit 40 that can supply the bio-fuel to the vehicle 2 of the system 1 for its operation. The bio-fuel can also be supplied from the supplying unit 40 to other vehicles 50 or units 50 that operate on fuel. Further, the other products such as fertilizers, chemicals etc. produced by the conversion unit 30 can be supplied to various units for their appropriate use.

An example below illustrates approximate figures (in numbers) for CO₂ generated by a small car, CO₂ captured by the algae to produce bio-mass, and bio-diesel produced from the bio-mass. A small car running for 20 km emits nearly 2.6 kg of CO₂. It has been found that 1 kg of dry algae can capture and consume around 1.8 kg of CO₂ at the ambient condition if suitable strains of algae are used. Further, 1 kg of dry algal biomass produced from an elite/superior strain of algae can produce about 30-40% lipids/oil which can be converted into about 300 ml of algal bio-diesel.

According to the present subject matter, the system 1 (FIG. 1) and the flow diagram (FIG. 2) illustrate an advantageous way to capture CO₂, from the atmosphere and emitted by the polluting units, for example vehicles, and convert the captured CO₂ to bio-fuel and other products such as fertilizers, chemicals etc.

Other advantages of the inventive carbon dioxide capture box and system will become better understood from the description and claims of an exemplary embodiment of such a unit.

The inventive carbon dioxide capture box and system of the present subject matter is not restricted to the embodiments that are mentioned above in the description.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

1-31. (canceled)

32. A system comprising:
a first unit that generates a polluted air comprising carbon dioxide;
a second unit mounted on the first unit, wherein the second unit comprises:
a solution comprising at least one alga suspended in a solvent to purify the polluted air, wherein the solution comprising the at least one alga is agitated upon operation of the first unit; and
a channel to guide the polluted air from the first unit to the second unit.

33. The system as claimed in claim 32, wherein the second unit comprises at least one purifying unit partially filled with the solution comprising the at least one alga.

34. The system as claimed in claim 33, wherein the purifying unit comprises a first inlet connected to a first end of the channel to receive the polluted air from the first unit.

35. The system as claimed in claim 33, wherein the purifying unit comprises a second inlet to receive a polluted air comprising carbon dioxide from atmosphere.

36. The system as claimed in claim 33, wherein the purifying unit comprises:
a first outlet to discharge at least one from a purified air and vapour generated in the purifying unit; and
a second outlet to discharge the solution.

37. The system as claimed in claim 33, the purifying unit comprises at least one surface made of a transparent material to provide sunlight to the at least one alga.

38. The system as claimed in claim 33, wherein the purifying unit is made of a material selected from the group consisting of acrylic, polycarbonate, polyvinyl chloride, recycle plastic, polyethylene, nylon, fiber-glass.

39. The system as claimed in claim 32, wherein the first unit is selected from the group consisting of a motorized vehicle, a non-motorized vehicle, and a movable object.

40. The system as claimed in claim 36, wherein the purified air generated in the purifying unit is supplied to a passenger cabin of the first unit through an outlet of the purifying unit.

41. The system as claimed in claim 32, wherein the channel comprises a second end connected to an exhaust collector, wherein the exhaust collector is placed near a discharge end of the first unit that emits the polluted air.

42. A carbon dioxide capture box comprising:
at least one purifying unit, partially filled with a solution comprising at least one alga suspended in a solvent, to purify a polluted air, wherein the polluted air comprises carbon dioxide;
characterized in that,
the purifying unit is a housing that comprises a transparent surface to provide sunlight to the at least one alga; and
the carbon dioxide capture box is mounted on a vehicle, wherein the solution comprising the at least one alga agitates upon movement of the vehicle.

43. The carbon dioxide capture box as claimed in claim 42, wherein the carbon dioxide capture box comprises a channel to guide the polluted air into the at least one purifying unit, wherein the polluted air is emitted by the vehicle.

44. The carbon dioxide capture box as claimed in claim 43, wherein the channel is connected to an exhaust collector at a second end of the channel to collect the polluted air emitted by the vehicle.

45. The carbon dioxide capture box as claimed in claim 42, wherein the at least one purifying unit comprises a second inlet to receive the polluted air, wherein the polluted air is from atmosphere.

46. The carbon dioxide capture box as claimed in claim 42, wherein the at least one purifying unit comprises:
a first outlet to discharge at least one from a purified air and vapour, generated in the purifying unit; and
a second outlet to discharge the solution comprising the at least one alga.

47. The carbon dioxide capture box as claimed in claim 46, wherein the purified air is supplied to at least one passenger of the vehicle.

48. The carbon dioxide capture box as claimed in claim 42, wherein the at least one purifying unit is made of a material selected from the group consisting of acrylic, polycarbonate, polyvinyl chloride, recycle plastic, polyethylene, nylon, fiber-glass.

49. The carbon dioxide capture box as claimed in claim 42, wherein the vehicle is selected from the group consisting of a cycle, a rickshaw, a trolley, a cart, a scooter, a motorcycle, a car, a jeep, an SUV, a transport container, a bus, a truck, a ship.

50. A method comprising:
capturing a polluted air comprising carbon dioxide from a vehicle by a carbon dioxide capture box;
purifying the polluted air by the carbon dioxide capture box;
supplying a bio-mass produced in the carbon dioxide capture box to a receiving unit;
transferring the bio-mass from the receiving unit to a conversion unit, wherein the bio-mass in the conversion unit is converted to a useful product;
transferring the useful product to a supplying unit; and
supplying the useful product to at least one from the vehicle and other units.

51. The method as claimed in claim 50, wherein the useful product is at least one from a bio-fuel, a fertilizer and a chemical.
